(19) United States
(12) Patent Application Publication
(10) Pub. No.: US 2020/0037894 A1
(43) Pub. Date: Feb. 6, 2020

(54) A PATIENT-SPECIFIC REMOTE ISCHEMIC PRECONDITIONING SYSTEM WITH MULTI-LAYER FEEDBACK CONTROL UNIT

(71) Applicant: MacQuarie University, Sydney (AU)

(72) Inventors: Ashkan Javadzadegan, North Ryde (AU); Andy Yong, North Ryde (AU); Abouzar Moshfegh, North Ryde (AU)

(21) Appl. No.: 16/481,329

(22) PCT Filed: Jan. 25, 2018
(86) PCT No.: PCT/AU2018/000005

§ 371 Date: Jul. 26, 2019

(30) Foreign Application Priority Data
Jan. 27, 2017 (AU) 2017900239

(51) Int. Cl. A61B 5/0225; A61B 5/026; A61B 5/0255; A61B 5/1455

(52) U.S. Cl. CPC A61B 5/0225; A61B 5/026; A61B 5/6824; A61B 5/1455; A61B 5/0255

(57) ABSTRACT

The technology relates to a remote ischemic preconditioning system having a cuff configured to contract about a limb of a subject; an actuator connected to the cuff that, when actuated, causes the cuff to contract about the limb of the subject; a controller that controls the actuator to operate according to a treatment protocol that includes a plurality of treatment cycles of contracting and releasing the cuff about the limb of a subject; a first sensor for measuring oxygen saturation level in the blood of the limb; a second sensor for measuring a pulse property in the limb; and a feedback control unit in communication with the controller and configured to receive the oxygen saturation measurement from the first sensor and the pulse property from the second sensor; wherein the feedback control unit is further configured to: compare the oxygen saturation level to a first predetermined value and signal the controller to operate the actuator to further inflate the cuff if the oxygen saturation level is above the predetermined value; and compare the pulse property to a second predetermined value and signal the controller to operate the actuator to further inflate the cuff if the pulse rate or pulse strength is above the predetermined value.

### TECHNICAL FIELD

The technology relates to systems for performing remote ischemic preconditioning of a subject. In particular the technology relates to systems having feedback control units to facilitate continual adjustment of the system to maintain ischemia.

### BACKGROUND

Ischemic heart disease is a significant cause of mortality and morbidity and it is known that tissue damage results from ischemia (where the blood supply is stopped) followed by reperfusion (when the blood flow is reestablished to the ischemic region). Ischemia reperfusion injury is caused by disturbances in the microcirculation and organs such as the kidney, heart, liver, pancreas, lung, brain and intestine are known to sustain damage following ischemia and reperfusion.

Ischemic preconditioning can mitigate ischemia reperfusion injury. During ischemic preconditioning part of a patient is subjected to a cycle of brief ischemia followed by reperfusion. This provides resistance to injuries caused subsequent to ischemic-reperfusion episodes, such as when a blocked artery is cleared allowing blood flow to return to a previously ischemic region. Ischemic preconditioning is generally accepted to be an important innate, protective mechanism against ischemia reperfusion injury.

Remote ischemic preconditioning refers to cycles of brief ischemia followed by reperfusion at a position remote from the tissue or organ to be protected. For example, remote ischemic preconditioning may involve inducing transient ischemia in a limb to protect the heart, kidney or brain. Remote ischemic preconditioning of a coronary artery territory has been shown to induce remote areas of the myocardium to be resistant to injury following prolonged ischemia.

Remote ischemic preconditioning has been effected using a sphygnamometer. The cuff of the sphygnamometer is placed about the patient's arm and inflated to a pressure that occludes blood flow through the arm (the ischemic pressure—i.e., a pressure typically greater than the patient's systolic blood pressure). The cuff remains inflated for a period of time specified by a doctor (the ischemic duration). After the ischemic duration, the pressure in the cuff is released to allow reperfusion of the limb for a period of time (the reperfusion duration). This cycle is then immediately repeated a number of times as specified by a doctor.

Using a sphygnamometer or other manual type tourniquet to perform remote ischemic preconditioning has a number of problems including that once the cuff is inflated to the ischemic pressure there is no way of determining whether or not the pressure needs to be adjusted during the ischemic period to account for changes in the patient's blood pressure. In addition, methods using a sphygnamometer do not monitor ischemia and so the exact ischemic duration is unknown.

### SUMMARY

Embodiments of the present invention relate to an automated system to perform patient-specific remote ischemic preconditioning that is responsive to changes in oxygen saturation level of the blood, pulse rate/strength in a limb such as a finger, pulse rate/strength in an ischemic limb as well as lactate acid level. The system typically does not require monitoring or intervention by a user to perform remote ischemic preconditioning. In addition, the system allows minimum pressure required to achieve adequate ischemic preconditioning is exerted on subject's limb in order to minimise pain and discomfort in the subject.

In a first aspect there is provided a remote ischemic preconditioning system comprising:

a cuff configured to contract about a limb of a subject;

an actuator connected to the cuff that, when actuated, causes the cuff to contract about the limb of the subject;

a controller that controls the actuator to operate according to a treatment protocol that includes a plurality of treatment cycles of contracting and releasing the cuff about the limb of a subject;

a first sensor for measuring oxygen saturation level in the blood of the limb;

a second sensor for measuring a pulse property in the limb; and

a feedback control unit in communication with the controller and configured to receive the oxygen saturation measurement from the first sensor and the pulse property from the second sensor;

wherein the feedback control unit is further configured to:
compare the oxygen saturation level to a first predetermined value and signal the controller to operate the actuator to further inflate the cuff if the oxygen saturation level is above the predetermined value; and
compare the pulse property to a second predetermined value and signal the controller to operate the actuator to further inflate the cuff if the pulse rate or pulse strength is above the predetermined value.

In embodiments, the limb is arm or leg of the subject. In several instances the limb is an arm of the subject.

In embodiments, the second sensor is arranged to measure the pulse property in a finger of the subject. Alternatively, the second sensor may be arranged to measure the pulse property at another area of the limb. In some instances the second sensor may be located in proximity of the cuff. The pulse property may comprise pulse strength or pulse rate.

In some embodiments, the first sensor is a pulse oximeter. The second sensor may be selected from a group consisting of a heart rate sensor, a photoplethysmographic sensor, an ultrasonic flow sensor, an infrared detector, and a near infrared sensor.

The second sensor may be a heart rate sensor.

In some embodiments, the system further comprises a sphygnamometer.

In embodiments, the system further comprises an ischemia pressure unit in communication with the controller and the feedback control unit; the ischemia pressure unit being arranged to determine a minimum ischemia pressure required to pass all feedback loops and therefore to minimise discomfort of the subject.

The ischemia pressure unit may be further arranged to receive values of subject-specific systolic and diastolic pressures; wherein the value of systolic pressure or average value of the systolic and diastolic pressures are used as the initial ischemia pressure and, if the initial pressure is sufficient to maintain ischemia, the ischemia pressure unit reduces the pressure until the minimum pressure required to pass all feedback loops and maintain ischemia. Furthermore, the ischemia pressure unit may be configured to continually or intermittently communicate with the controller and the feedback control unit.

In embodiments, the feedback control unit is configured to continually or intermittently communicate with the controller.

In embodiments, the system further comprises a third sensor. The third sensor may be arranged to measure pulse rate or pulse strength in the ischemic limb and the feedback control unit is configured to receive the pulse rate or pulse strength in the ischemic limb from the third sensor and compare the pulse rate or pulse strength to a third predetermined value, wherein, if the pulse rate or pulse strength is above the predetermined value, the feedback control unit signals the controller to operate the actuator to further inflate the cuff.

In embodiments, the system further comprises a fourth sensor. The fourth sensor may be a lactic acid sensor; wherein the feedback control unit is configured to a receive lactic acid level from the fourth sensor and compare the lactic acid level to a fourth predetermined value, wherein, if the lactic acid level is below the predetermined value, the feedback control unit signals the controller to operate the actuator to further inflate the cuff.

In a second aspect there is provided a method of performing remote ischemic preconditioning of a subject, the method comprising:
providing the system in accordance with the first aspect;
attaching the cuff about a limb of a subject;
activating the controller to operate according to a treatment protocol that includes a plurality of treatment cycles of contracting and releasing the cuff about the limb of a subject wherein each treatment cycle comprises:
an ischemic duration, during which the controller receives signals from the at least one of the sensors to maintain the cuff contracted about the limb to occlude blood flow through the limb thereby maintaining ischemia, and
a reperfusion duration, during which the cuff is maintained in an at least partially relaxed state to allow blood flow through the limb.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

In order that the present invention may be more clearly understood, embodiments of the invention will be described with reference to the drawing.

### BRIEF DESCRIPTION OF THE DRAWING

FIG. 1 is a flow chart of one embodiment of an operating scheme of the remote ischemic preconditioning system.

FIG. 2 is a flow chart of one embodiment of an operating scheme of the remote ischemic preconditioning system

FIG. 3 is a flow chart of one embodiment of an operating scheme of the remote ischemic preconditioning system with a three layer FCU (FCU(I)).

FIG. 4 is a flow chart of one embodiment of an operating scheme of the remote ischemic preconditioning system with a three layer FCU (FCU(II)).

FIG. 5 is a flow chart of one embodiment of an operating scheme of the remote ischemic preconditioning system with a three layer FCU (FCU(III)).

FIG. 6 is a flow chart of one embodiment of an operating scheme of the remote ischemic preconditioning system with a four layer FCU (FCU(I)).

FIG. 7 is a flow chart of one embodiment of an operating scheme of the remote ischemic preconditioning system with a four layer FCU (FCU(II)).

FIG. 8 is a flow chart of one embodiment of an operating scheme of the remote ischemic preconditioning system with a four layer FCU (FCU(III)).

FIG. 9 is a flow chart of an embodiment of an operating scheme of the remote ischemic preconditioning system that was implemented on 12 patients.

FIG. 10 shows the monitored pulse oximetry plethysmographic waveform during the RIPC treatment of a representative patient for different values of the systolic pressure.

### DESCRIPTION

The systems described herein can be used to provide a safe and reliable method of performing remote ischemic preconditioning. The system is used to execute a treatment protocol designed by a physician or other medical professional and can execute that protocol with no or minimal oversight by highly qualified medical personnel. The systems described herein are designed for delivering a subject-specific remote ischemic preconditioning (RIC) treatment. The system typically comprises two units: a treatment unit (TU) and feedback control unit (FCU). Both units communicate throughout treatment.

### Treatment Unit

As used herein Treatment Unit and Treatment Protocol Unit are used interchangeably.

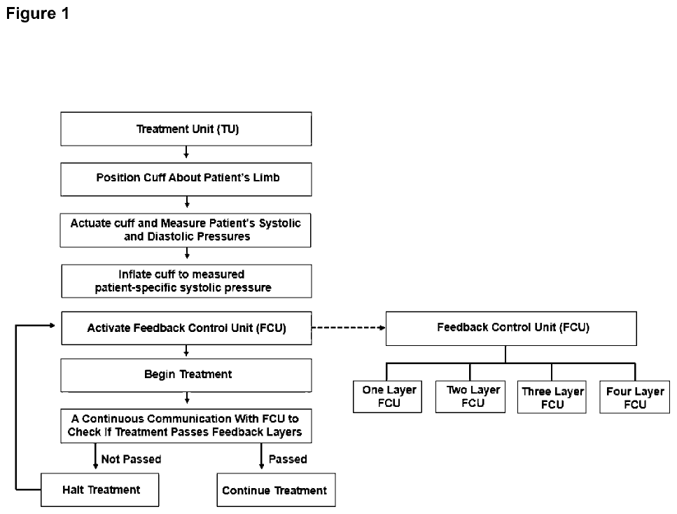

The flowchart in FIG. 1 provides an illustration of one embodiment of the system and how it is used to perform remote ischemic preconditioning. Initially, the cuff of the treatment unit is placed about a limb of the subject, typically an arm or a leg. The cuff is connected to an actuator and when the system is activated the actuator causes the cuff to contract about the limb of the subject.

In one embodiment, when the system is activated, the cuff inflates to pressure of about 220 mmHg and then starts to deflate slowly until subject's systolic pressure is measured, this is followed by further deflation of the cuff until subject's diastolic pressure is measured. In some embodiments the system includes a display screen, such as an LCD screen, and both the measured systolic and diastolic pressures are displayed.

In some embodiments, the cuff is part of a sphygnamometer and in these embodiments once the cuff contracts about the limb of the subject systolic and diastolic pressures can be measured and the cuff inflated to exert an initial pressure on the limb that is substantially the same as or greater than the measured systolic pressure.

For example, the cuff may be inflated to exert an initial pressure on the limb that is about 1%, 2.5%, 5%, 7.5%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5% or 80% greater than the measured systolic pressure. In one embodiment the cuff may be inflated to exert an initial pressure on the limb that is about 120 mm Hg, or about 130 mm Hg, or about 140 mm Hg, or about 150 mm Hg, or about 160 mm Hg, or about 170 mm Hg, or about 180 mm Hg, or about 190 mm Hg, or about 200 mm Hg, or about 210 mm Hg, or at least about 220 mm Hg.

Typically the cuff is inflated to exert pressure on the limb that is greater than the measured systolic pressure. However, in other embodiments, the pressure exerted by the cuff and required for occluding blood flow to the limb is not necessarily greater than systolic pressure, for example in patients with stage 1 or 2 hypertension.

In one embodiment, the TU comprises an LCD display, analog heart beat sensor, pressure sensor (MPXV5050GP), microcontroller (PIC16F886 & PIC16F630), remote controller (Ev1527), voltage regulator (LM217), transistors (C945 & 2SD880), a quad operational amplifier (LM324N) and 2 electronic air flow control valves. Power is supplied to the circuit of this embodiment by batteries, such nickel-metal hydride batteries.

The cuff is configured to be positioned about the limb of a subject and to contract about the limb when actuated. In one embodiment, the cuff is positioned about a subjects upper arm, calf, or thigh and remains in place about the limb. For example the cuff may be fastened to itself by way of a hook and loop type material so the cuff does not dislodge from the subject's limb before actuation. On activation of the system, the actuator causes inflation of the cuff to about 220 mm Hg. This pressure is applied around the limb such that blood flow to the portion of the limb distal to the cuff is constricted.

The cuff typically includes an inflatable bladder adapted to receive a fluid, such as air. The fluid causes the cuff expand and exert pressure on the subject's limb. The bladder is typically constructed of a material that is substantially fluid impermeable such as silicone or rubber. The bladder comprises a port to allow fluid to enter and exit the bladder. The port may be in communication with a conduit such as an air hose that facilitates a connection to the actuator. The conduit may be attached to the port for example by way of a threaded or clip-in coupling. In some embodiments the cuff itself may be substantially fluid impermeable and act as the bladder. In another embodiment, a plurality of inflatable bladders may be incorporated into a single cuff.

To account for variations in subject size some embodiments provide a cuff that is adjustable to fit a number of different limb girths. In some embodiments, the cuff comprises an inflatable sleeve having a length of about 1.0 m, 0.75 m, 0.5 m, 0.25 m, 0.2 m or about 0.15 m. These lengths are to allow the cuff to be used with subjects of all sizes including larger or obese subjects, as well as infants. For example, the sleeve suitable for use with a newborn may be sufficient to surround an limb with a circumference of about 6 cm. In other embodiments the sleeve may be sufficient to surround a limb with a circumference of about 6-15 cm (e.g. an infant), 16-21 cm (e.g. a child), 22-26 cm (e.g. an adolescent or small adult), 27-34 cm (an adult), 35-44 cm (a large adult), or 45-52 cm (for example an adult thigh).

It is anticipated that any device known in the art suitable for inflating or deflating a bladder may be used as an actuator which, when actuated, causes the cuff to inflate and thereby apply pressure about a subject's limb. In one embodiment the actuator comprises a pump to apply pressurised air to the cuff via a hose. The actuator can also comprise at least one valve that, when opened, allows air to flow between the pump and the cuff. In addition there may be a release valve that, when opened, allows the pressurised air to escape the cuff so that the cuff loosens about the subject's limb. In some embodiments there may be a single valve that controls the flow of air into and out of the cuff. In one embodiment the actuator is an air flow control valve.

In some embodiments the release valve may be a valve that can be actuated to open (or close) quickly to allow air to quickly be released from the cuff. An example of a suitable valve is a solenoid In other embodiments the release valve may be actuated to open or close slowly, for example to allow adjustment of the pressure of the cuff or to allow a more controlled release of pressure such as may be required when the subject's blood pressure is measured or in embodiments where the pressure is adjusted in response to the FCU.

In some embodiments there may be a single valve that controls the flow of air into and out of the cuff.

In some embodiments the system includes a controller that controls the actuator to operate the system according to a treatment protocol that includes a plurality of treatment cycles. The controller receives information for treatment protocol and receives information from the FCU to control the actuator to perform remote ischemic preconditioning.

The controller can implement a treatment protocol (see below) in any number of ways. For example, the controller can implement the treatment protocol using hardware, software or a combination thereof. In embodiment where the treatment protocol is implemented in software the software code can be executed by a processor or collection of processors, whether those processors are in a single device (such as a computer or the controller) or distributed among multiple devices (for example the controller and a mobile device in communication with the controller). In one embodiment, the controller includes a link to communicate via a cable or wirelessly to a remote location. The function of the controller can be implemented in any number of ways. For example the system may include a dedicated hardware controller or the controller may take the form of one or more processors programmed to perform the functions set out above.

Implementation of a treatment protocol typically comprises at least one computer-readable medium (e.g., computer memory or usb storage device) encoded with a treatment protocol as a computer program (i.e., a plurality of instructions). When the program is executed by the controller the treatment protocol is implemented. In some embodiments the computer-readable medium is adapted to be transportable such that the stored treatment protocol can be loaded onto any computer system to implement the treatment protocol. It is anticipated that any type of computer code can be employed to program a processor to implement the treatment protocol.

### Feedback Control Unit (FCU)

The Feedback Control Unit (FCU) is in communication with the controller and once the cuff is inflated the FCU receives measurements from at least one sensor. For example, in one embodiment the FCU is configured to receive an oxygen saturation measurement from a sensor and compare the oxygen saturation level to a predetermined value. If the oxygen saturation level is above a predetermined value the feedback control unit signals the controller to operate the actuator to further inflate the cuff. Similarly the FCU can be configured to receive an additional measurement, for example a pulse rate or pulse strength measurement from a sensor and compare the pulse rate or pulse strength to a predetermined value. If the pulse rate or pulse strength is above a predetermined value the feedback control unit signals the controller to operate the actuator to further inflate the cuff.

The FCU can be configured to receive the measurements sequentially or at the same time. In some embodiments the FCU compares one measurement to a predetermined value and only when the measurement matches or exceeds the predetermined value or range will the FCU compare another measurement to another predetermined value or range.

The FCU can be a one layer, a two layer, a three layer or a four layer feedback loop. In the one layer feedback loop, the FCU is equipped with a single sensor whereas in two, three and four layer feedback loops, the FCU is equipped with two, three and four sensors, respectively.

The sensors may measure oxygen saturation, blood flow, heart rate, pulse rate, pulse strength, temperature or lactic acid level in a portion of the subject. Typically the sensors take measurements from the limb that is being treated.

The sensors that can be used to confirm the cessation of blood flow include but not limited to pulse oximeter, photoplethysmographic transducer, ultrasonic flow transducer, temperature transducer, infrared detector, near infrared transducer or lactic acid sensor.

In one embodiment the one layer FCU comprises a first sensor for measuring oxygen saturation of the blood of the limb. The FCU will compare the oxygen saturation to a predetermined value and if the oxygen saturation is above the predetermined oxygen saturation value the FCU sends a signal to the controller to increase the contraction of the cuff until the oxygen saturation is at or below the predetermined oxygen saturation value. For example the predetermined blood oxygen saturation level percentage (SpO2) can be 90%, 80%, 70%, 60%, 50%,40%, 30%, 20%, or 10%.

The two layer FCU comprises a second sensor, for example that identifies pulse rate or pulse strength, for example in a finger. The FCU will compare the pulse rate or pulse strength to a predetermined value and if the pulse rate or pulse strength is above the predetermined value the FCU sends a signal to the controller to increase the contraction of the cuff until the pulse rate or pulse strength is at or below the predetermined pulse rate or pulse strength values. For example the predetermined pulse rate or pulse strength values in voltage are 8, 7, 6, 5, 4, 3, 2, 1. Alternatively the predetermined values for pulse rate in bpm are 120, 110, 100, 90, 80, 70, 60, 50, or 40.

The three layer FCU comprises a third sensor, for example a sensor that identifies pulse rate or pulse strength in an ischemic limb. The FCU compares the pulse rate or pulse strength to a predetermined value and if the pulse rate or pulse strength is above the predetermined pulse rate or pulse strength value the FCU sends a signal to the controller to increase the contraction of the cuff until the pulse rate or pulse strength is at or below the predetermined pulse rate or pulse strength value. For example the predetermined pulse rate or pulse strength values in voltage are 8, 7, 6, 5, 4, 3, 2, 1.

The four layer FCU comprises a fourth sensor, for example a sensor that measures the lactic acid level. The FCU will compare the lactic acid level to a predetermined value and if the lactic acid level is below the predetermined lactic acid level the FCU sends a signal to the controller to increase the contraction of the cuff until the lactic acid level is at or above the predetermined lactic acid level. For example, the predefined lactic acid level acid level is 1, 1.5, 2, 2.5, 3, 3.5, 4, 4.5, 5, 5.5, 6, 6.5, 7, 7.5, 8, 8.5, or 9 mMol/L.

In the case of a two layer FCU, oxygen saturation is measured in the first layer and in the second layer pulse rate or pulse strength is measured, typically in the finger. Once the cuff is inflated, the first layer becomes active and the level of blood oxygen is measured. If the oxygen level is above a predefined value, the FCU sends a signal to the controller and the pressure within cuff incrementally increases so as to further reduce the blood flow to the limb. Once the oxygen saturation level reaches or falls below the predefined value layer 2 is activated. In layer 2 the second sensor measures pulse rate or pulse strength (for example in the finger) to ensure that it is below a predefined value. If the pulse rate or pulse strength is above a predefined value, the FCU sends a signal to the controller and the pressure within cuff incrementally increases so as to further reduce the blood flow to the limb.

In the instance of a three layer FCU, oxygen saturation is measured in the first layer, pulse rate or pulse strength in the finger is measured in the second layer and in the third layer pulse rate or pulse strength in the limb is measured. Once the cuff is inflated the first layer becomes active and the level of blood oxygen is measured. If the oxygen level is above a predefined value, the FCU sends a signal to the controller and the pressure within cuff incrementally increases so as to further reduce the blood flow to the limb. Once the oxygen saturation level reaches or falls below the predefined value layer 2 is activated. In layer 2 the second sensor measures pulse rate or pulse strength in the finger to ensure that it is below a predefined value. If the pulse rate or pulse strength is above a predefined value, the FCU sends a signal to the controller and the pressure within cuff incrementally increases so as to further reduce the blood flow to the limb. Once the pulse rate or pulse strength value in the finger reaches or falls below the predefined value layer 3 is activated. In layer 3 the third sensor measures pulse rate or pulse strength in the limb to ensure that it is below a predefined value. If the pulse rate or pulse strength is above a predefined value, the FCU sends a signal to the controller and the pressure within cuff incrementally increases so as to further reduce the blood flow to the limb.

In one embodiment of a four layer FCU, oxygen saturation is measured in the first layer, pulse rate or pulse strength in the finger is measured in the second layer, pulse rate or pulse strength in the limb is measured in the third layer and in the fourth layer lactate acid level is measured. Once the cuff is inflated the first layer becomes active and the level of blood oxygen is measured. If the oxygen level is above a predefined value, the FCU sends a signal to the controller and the pressure within cuff incrementally increases so as to further reduce the blood flow to the limb. Once the oxygen saturation level reaches or falls below the predefined value layer 2 is activated. In layer 2 the second sensor measures pulse rate or pulse strength in the finger to ensure that it is below a predefined value. If the pulse rate or pulse strength is above a predefined value, the FCU sends a signal to the controller and the pressure within cuff incrementally increases so as to further reduce the blood flow to the limb. Once the pulse rate or pulse strength value in the finger reaches or falls below the predefined value, then layer 3 is activated. In layer 3, the third sensor measures pulse rate or pulse strength in the limb to ensure that it is below a predefined value. If the pulse rate or pulse strength is above a predefined value, the FCU sends a signal to the controller and the pressure within cuff incrementally increases so as to further reduce the blood flow to the limb. Once the pulse rate or pulse strength value in the limb reaches or falls below the predefined value, then layer 4 is activated. In layer 4, the fourth sensor measures lactate acid level to ensure that it is above a predefined value. If the lactate acid level is below a predefined value, the FCU sends a signal to the controller and the pressure within cuff incrementally increases so as to further reduce the blood flow to the limb.

In one embodiment the FCU contains an LCD display, a microcontroller (PIC18F2520), a pulse oximetry sensor (MAX30100), a light emitting diode (IR), and a light emitting diode (RED).

The cuff pressure required for passing all feedback layers is the ischemia pressure. In some embodiments the oxygen saturation and pulse rate or pulse strength and the lactic acid level are constantly displayed, for example on an LCD screen.

The ischemic pressure will be above the subjects systolic blood pressure. For example the ischemic pressure may be about 5 mm Hg, 10 mm Hg, 15 mm Hg, 20 mm Hg, 25 mm Hg, 30 mm Hg, 35 mm Hg, 40 mm Hg, 45 mm Hg, or at least about 50 mm Hg above the subject's systolic pressure. In other embodiments, the ischemic pressure may be at least 102%, 105%, 110%, 115%, 120%, 125%, 130%, 135%, 140%, 145%, 150%, 155%, 160%, 165%, 170%, 175%, or at least 180% of the subject's systolic blood pressure.

During the treatment protocol the FCU constantly or intermittently communicates with the TU to ensure that the ischemia pressure is sufficient to occlude the blood flow in the subject's limb for example because they are nervous, moving, reacting to stimuli such as an injection, reacting to pain caused by applied pressure on the limb, taking medicine, the ischemia pressure initially established for occluding blood flow and creating ischemia may not be sufficient. The advantage of constant or intermittent monitoring is that if the initially established ischemia becomes insufficient the signals from the sensors of the FCU will be received by the TU which activate the actuator to adjust the pressure in the cuff in order to maintain the ischemia pressure. In this way ischemia pressure is maintained throughout the treatment resulting in more efficient preconditioning.

The FCU may also include other sensors, for example to receive information on the system, such as air pressure within the cuff or the pressure applied by the cuff. For example the cuffs may include a pressure sensor to measure pressure within the cuff or the pressure the cuff is exerting on the limb. Typically cuff pressure is used as a direct indication of blood pressure of the limb surrounded by the cuff. The controller can be programmed to establish a specific cuff pressure for the ischemic duration of a treatment cycle. In some embodiments the pressure sensor is positioned within the bladder of the cuff, in the air hose or in the actuator. A pressure sensor may be positioned on an inner surface of the cuff to directly measure the pressure between the cuff and an outer surface of the subject's limb.

### Treatment Protocol

Typically the treatment protocol consists of a plurality of treatment cycles. Each cycle comprises a ischemia duration and a reperfusion duration. The ischemia duration begins once the ischemia pressure is reached. Typically the ischemia pressure is sufficient to completely occlude blood flow in the subject's limb. The ischemia duration ends once the pressure to the cuff is released. Once pressure to the cuff is released blood flow returns to the subject's limb and the reperfusion duration begins. The treatment cycle involves cuff actuation to inflate the cuff about the limb of a subject to occlude blood flow in the limb.

Typically the user defines a treatment protocol by entering details (e.g. ischemic duration, reperfusion duration and number of treatment cycles) into the system, typically the details are entered in the controller. For example, the controller receives instructions for treatment protocol, such as total treatment duration in minutes, ischemia duration in minutes and reperfusion duration in minutes.

The ischemic duration may be from about a few seconds to at least about 30 minutes. For example the ischemic duration may be about 5 seconds, about 10 seconds, about 20 seconds, about 30 seconds, about 40 seconds, about 50 seconds or about a minute. The ischemic duration may be about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or at least about 60 minutes.

In some embodiments, the ischemic duration may vary between treatment cycles in one treatment protocol and in other embodiments, the ischemic duration remains constant.

The controller acts, with feedback from the FCU, to maintain the ischemic pressure without the need for external monitoring by a user. Accordingly in some embodiments the cuff pressure may be reduced or increased within the ischemic duration to maintain the ischemic pressure. That is the FCU can cause the cuff pressure to vary to maintain the ischemic pressure.

In some embodiments, the treatment protocol includes measurement of the subject's systolic blood pressure, diastolic blood pressure or both. Identification or measurement of systolic or diastolic blood pressure may occur at any time during a treatment protocol. In some embodiments the subject's systolic blood pressure is measured at the start of each treatment cycle. In other embodiments, systolic pressure is measured only once during the treatment protocol. In other embodiments, systolic pressure is measured as the cuff is released at the end of the ischemic cycle. In other embodiments the treatment protocol may be performed without measuring the systolic pressure.

Pressure to the cuff is released at the end of the ischemic duration. Typically the cuff pressure is reduced to a point below diastolic pressure.

The reperfusion duration follows cuff release. In some embodiments the reperfusion duration may be from about a few seconds to at least about 30 minutes. For example the ischemic duration may be about 5 seconds, about 10 seconds, about 20 seconds, about 30 seconds, about 40 seconds, about 50 seconds or about a minute. The reperfusion duration may be about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or at least about 60 minutes.

The treatment protocol comprises a plurality treatment cycles. In one embodiment the treatment protocol may comprise one treatment cycle repeated a number of times. Alternately, the treatment protocol may comprise one or more cycles that differ from another cycle in the protocol. For example the cycles may differ by ischemic duration or reperfusion duration.

In some embodiments there may be 2, 3, 4, 5, 6, 7, 8, 9 or 10 treatment cycles.

### Embodiments of the System

Additional embodiments of the system are described as follows with reference to the Figures.

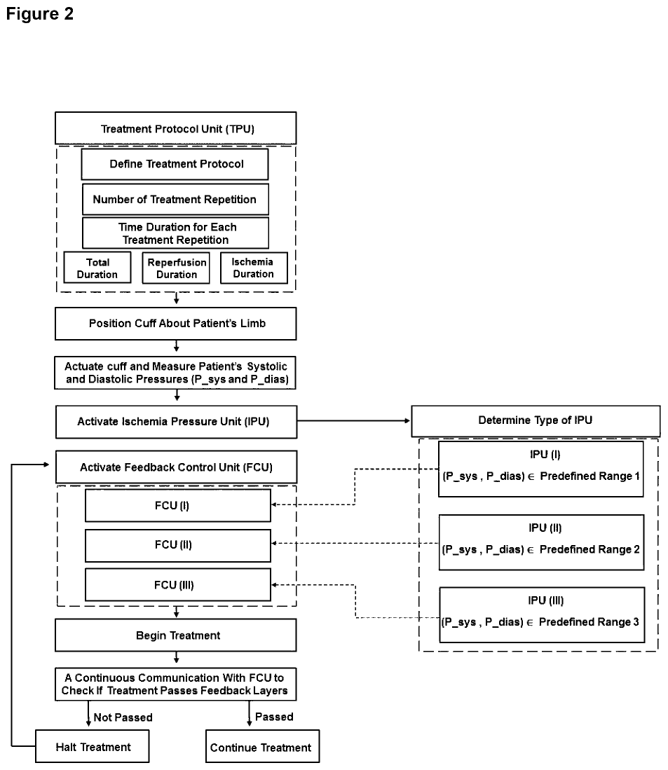

The flowchart in FIG. 2 is an illustration of a remote ischemic preconditioning system. As shown, the system consists of 3 main units (shown by dashed rectangles):

Treatment Protocol Unit (TPU)

Ischemia Pressure Unit (IPU)

Feedback Control Unit (FCU)

The TPU is used to define the treatment protocol by entering 4 main parameters, as follows:

1. Number of Treatment Repetition

2. Total Duration of Each Treatment

3. Ischemia Duration for Each Treatment

4. Reperfusion Duration for Each Treatment

The IPU is used to minimise patient's pain and discomfort by determining minimum pressure required to maintain ischemia. Further, the IPU is used to deliver a patient-specific RIC treatment by classifying the measured systolic and diastolic pressures.

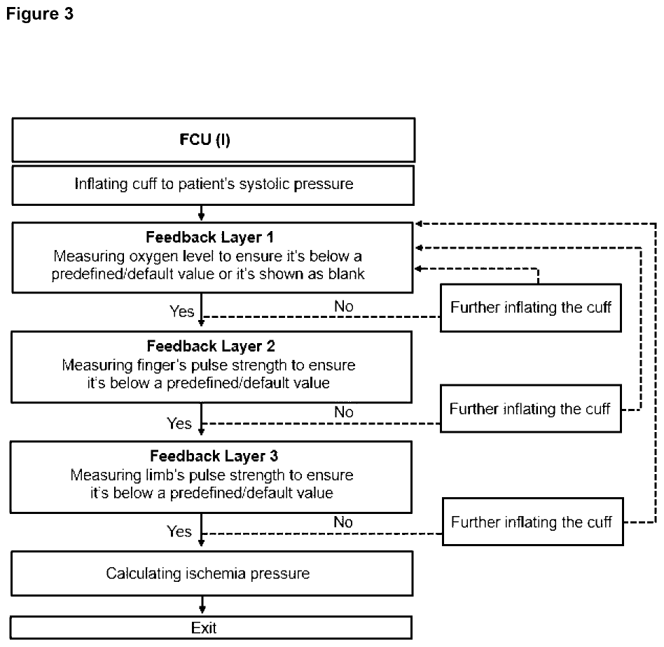

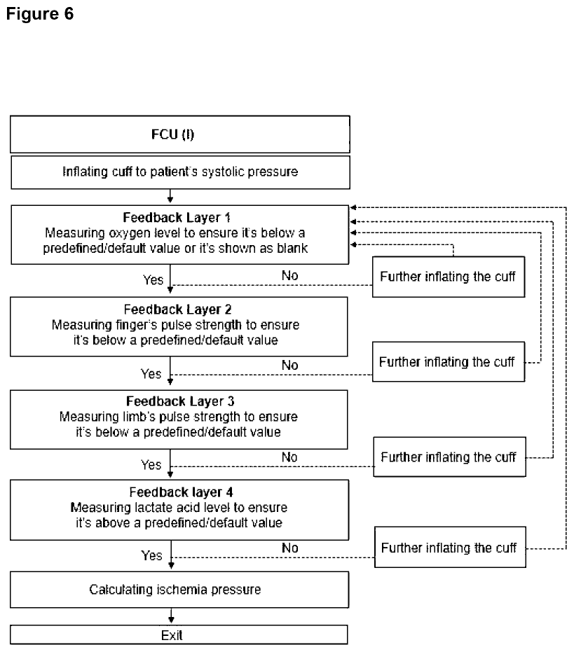

The FCU is a multilayer feedback loop to facilitate continual adjustment of the system to maintain ischemia. FIG. 3 to FIG. 5 are exemplary flowcharts of systems with a three layer FCU. FIG. 6 to FIG. 8 are exemplary flowcharts of systems with a four layer FCU.

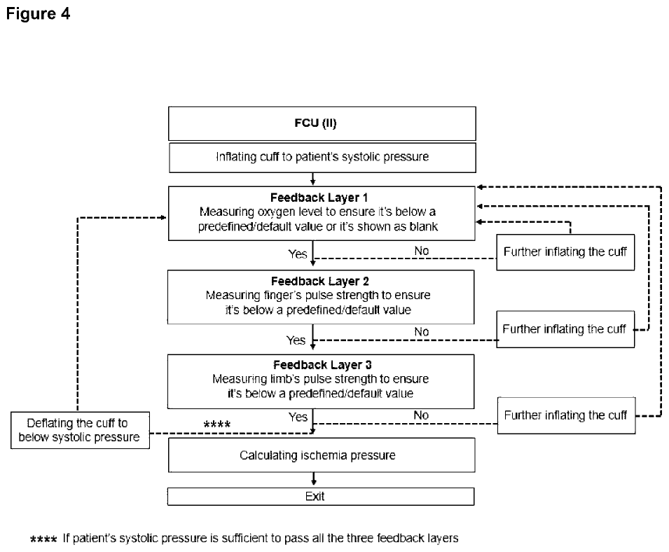

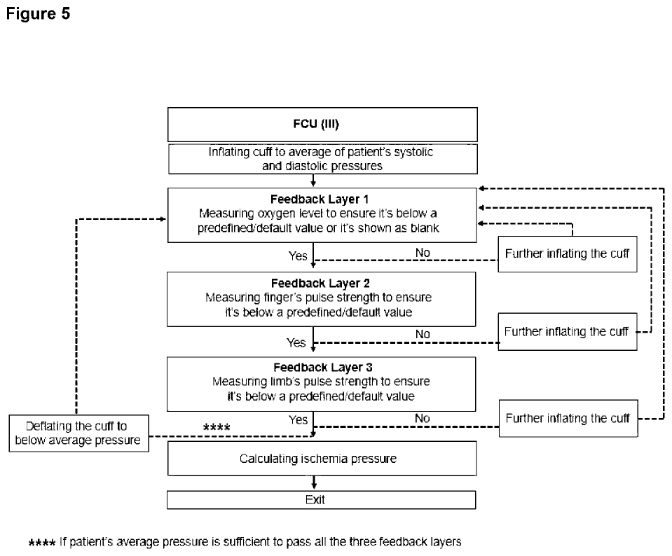

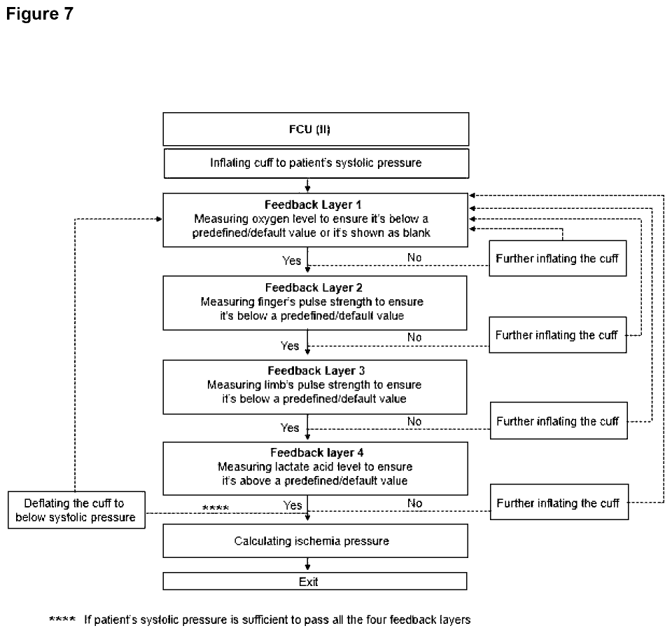

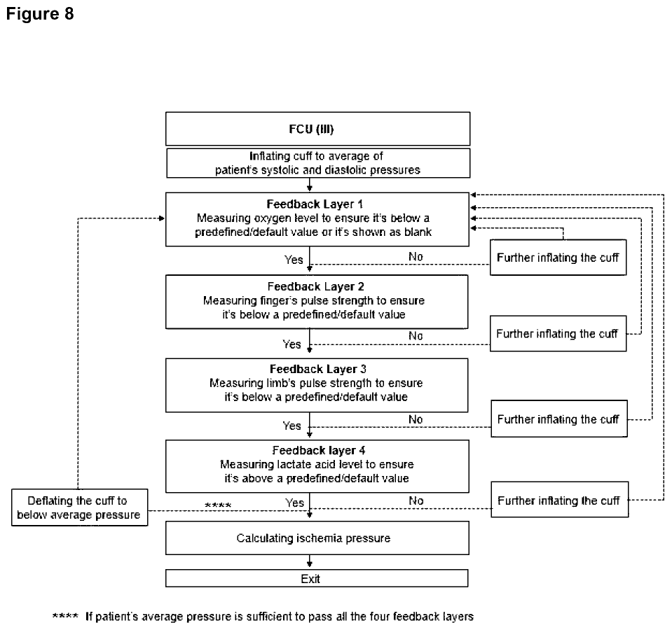

The system illustrated in FIG. 2 can be used as follows:
1. Define the treatment protocol via TPU.
2. Position the cuff about patient's limb and measure systolic and diastolic pressures.
3. Use the measured systolic and diastolic pressures to define the type of IPU: IPU (I), IPU (II), IPU (III) wherein IPU (I) is for patients whose systolic and diastolic pressures (P_sys and P_dias) are within predefined range 1. The predefined range 1 can be defined by medical professional or can be set as default values. An example of the default predefined range 1:
   P_sys<120 mmHg
   P_dias<80 mmHg
IPU (II) is for patients whose systolic and diastolic pressures (P_sys and P_dias) are within predefined range 2. The predefined range 2 can be defined by medical professional or can be set as default values. An example of the default predefined range 2:
   120=<P_sys<160 mmHg
   80=<P_dias<100 mmHg
IPU (III) is for patients whose systolic and diastolic pressures (P_sys and P_dias) are within predefined range 3. The predefined range 3 can be defined by medical professional or can be set as default values. An example of the default predefined range 3:
   P_sys >=160 mmHg
   P_dias>=100 mmHg
4. Activate the FCU based on the type of IPU
For IPU (I): Three-layer FCU (I) (FIG. 3) or Four-layer FCU (I) (FIG. 6)
For IPU (II): Three-layer FCU (II) (FIG. 4) or Four-layer FCU (II) (FIG. 7)
For IPU (III): Three-layer FCU (III) (FIG. 5) or Four-layer FCU (III) (FIG. 8)

In embodiments using a three layer FCU (I), for example as shown FIG. 3, the system operates as follows:
1: The cuff is inflated to measured patient's systolic pressure.
2: The feedback layer 1 comes into effect where the blood oxygen saturation level percentage (SpO₂) is measured by means of the pulse oximetry sensor. If SpO₂ is below a predefined/default value or if it is shown as blank, then it means that the feedback layer 1 is passed. Otherwise the cuff is further inflated to above the systolic pressure until the feedback layer 1 is passed.
3: The feedback layer 2 comes into effect where pulse rate or pulse strength in the finger is measured by means of the pulse oximetry sensor. If the pulse rate or pulse strength is below a predefined value then it means that the feedback layer 2 is passed. Otherwise the cuff is further inflated to above the systolic pressure until the feedback layer 2 is passed.
4: The feedback layer 3 comes into effect where the pulse rate or pulse strength in the limb is measured by means of the pressure sensor. If pulse rate or pulse strength is below a predefined value then it means that the feedback layer 3 is passed. Otherwise the cuff is further inflated to above the systolic pressure until the feedback layer 3 is passed.
5: If all three feedback layers are passed, then the ischemia pressure is calculated and the remote ischemic preconditioning treatment begins.

In embodiments using a three layer FCU (II), for example as shown FIG. 4, the system operates as follows:
1: The cuff is inflated to measured patient's systolic pressure.
2: The feedback layer 1 comes into effect where the blood oxygen saturation level percentage (SpO₂) is measured by means of the pulse oximetry sensor. If SpO₂ is below a predefined/default value of if it is shown as blank, then it means that the feedback layer 1 is passed. Otherwise, the cuff is further inflated to above the systolic pressure until the feedback layer 1 is passed.
3: The feedback layer 2 comes into effect where pulse rate in the finger is measured by means of the pulse oximetry sensor. If pulse rate is below a predefined value, then it means that the feedback layer 2 is passed. Otherwise the cuff is further inflated to above the systolic pressure until the feedback layer 2 is passed.
4: The feedback layer 3 comes into effect where pulse rate or pulse strength in the limb is measured by means of the pressure sensor. If pulse rate or pulse strength is below a predefined value, then it means that the feedback layer 3 is passed. Otherwise the cuff is further inflated to above the systolic pressure until the feedback layer 3 is passed.
5: If the patient's systolic pressure is enough for passing all three feedback layers, then the cuff is deflated to below the systolic pressure until all three layers are passed again. This step is to find the minimum pressure required to pass the all three layers and therefore to minimise the patient's pain and discomfort.
6: The calculated pressure to pass all three feedback layers is used to begin the remote ischemic preconditioning treatment.

In embodiments using a three layer FCU (III), for example as shown FIG. 5, the system operates as follows:
1: The cuff is inflated to average of patient's systolic and diastolic pressures:

Average Pressure=(Systolic Pressure+Diastolic Pressure)/2.
2: The feedback layer 1 comes into effect where the blood oxygen saturation level percentage (SpO₂) is measured by means of the pulse oximetry sensor. If SpO₂ is below a predefined/default value or if it is shown as blank, then it means that the feedback layer 1 is passed. Otherwise the cuff is further inflated to above the average pressure until the feedback layer 1 is passed.
3: The feedback layer 2 comes into effect where pulse rate or pulse strength in the finger is measured by means of the pulse oximetry sensor. If pulse rate or pulse strength is below a predefined value, then it means that the feedback layer 2 is passed. Otherwise the cuff is further inflated to above the average pressure until the feedback layer 2 is passed.
4: The feedback layer 3 comes into effect where pulse rate or pulse strength in the limb is measured by means of the pressure sensor. If pulse rate or pulse strength is below a predefined value, then it means that the feedback layer 3 is passed. Otherwise the cuff is further inflated to above the systolic pressure until the feedback layer 3 is passed.
5: If the patient's average pressure is enough for passing all three feedback layers, then the cuff is deflated to below the average pressure until all three layers are passed again. This step is to find the minimum pressure required to pass the all three layers and therefore to minimise the patient's pain and discomfort.
6: The calculated pressure to pass all three feedback layers is used to begin the remote ischemic preconditioning treatment.

In embodiments using a four layer FCU (I), a four layer FCU (II) and a four layer FCU (III), for example as shown respectively in FIG. 6, FIG. 7 and FIG. 8, the first three layers are similar respectively to the three-layer FCU (I), three-layer FCU (II) and three-layer FCU (III). However, in the fourth feedback layer, the level of lactic acid is measured using a lactate sensor such as BSXinsight sensor. The lactate sensor is equipped with an internal LED light-emitting device and a light detector. If it is above a predefined/default value, then it means that the feedback layer 4 is passed. Otherwise the cuff is further inflated until the feedback layer 4 is passed.

During the remote ischemic preconditioning treatment, there is a continuous communication with FCU to ensure that all four feedback layers are always passed throughout the treatment. If any layer of FCU is not passed during the treatment, then the treatment is halted, and the pressure in the cuff is changed until all feedback layers are passed.

### Use of the System

Remote Ischemic Preconditioning (RIC) was performed with on 12 patients with no prior history or symptoms of heart disease. The implemented FCU is a single layer feedback loop based on pulse oximetry plethysmographic waveform.

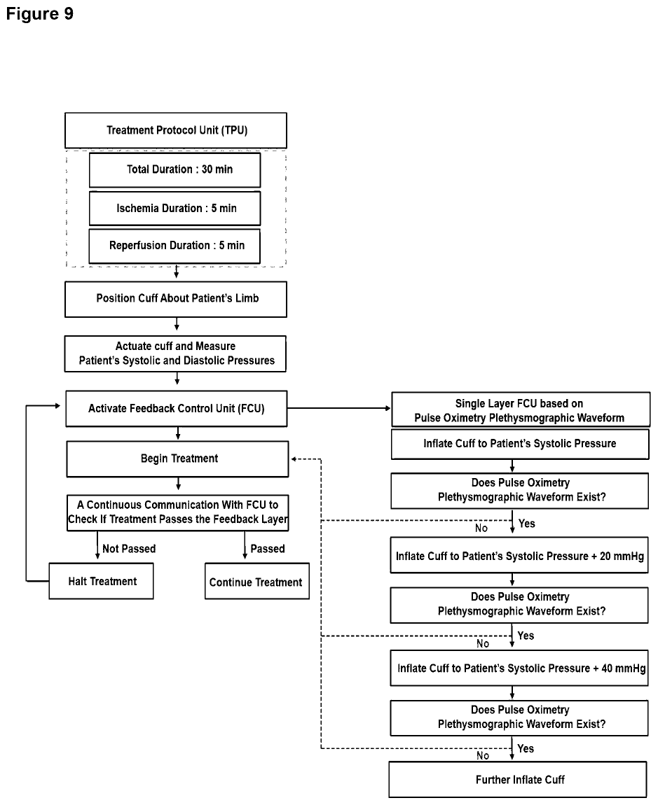

The flowchart in FIG. 9 illustrates how RIC was conducted. In brief, the systolic pressure of each patient was measured and the cuff inflated to measured systolic pressure. Then a single layer FCU is activated to check for the existence of a pulse oximetry plethysmographic waveform. If there is a waveform, then the cuff is inflated to 20 mmHg above the systolic pressure and the existence of pulse oximetry plethysmographic waveform is checked again. If there is still a waveform, then the cuff is inflated to 40 mmHg above the systolic pressure and the existence of plethysmographic waveform is checked again. This continues until there is no plethysmographic waveform.

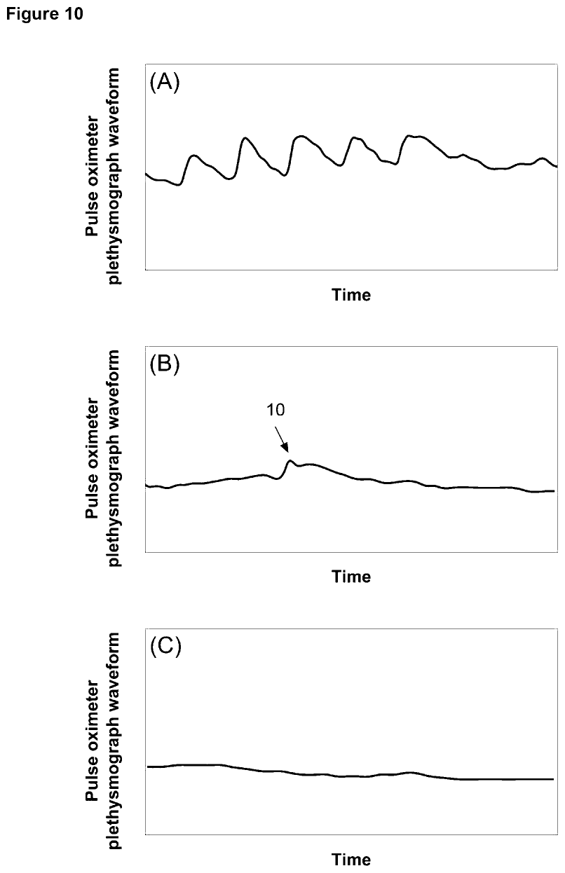

Referring now to FIG. 10, there is shown the monitored pulse oximetry plethysmographic waveform during the RIPC treatment of a representative patient. FIG. 10(A) shows that when the patient's systolic pressure is applied on his limb, the plethysmographic waveform still exists, indicating that a full ischemia is not yet achieved. FIG. 10(B) shows that, although by applying a higher pressure on the patient's limb (systolic pressure+20 mmHg), the plethysmographic waveform begins to disappear, some small waves 10, still exists. This again indicates that this pressure (systolic pressure+20 mmHg) is also not enough to achieve the full ischemia. However, FIG. 10(C) shows that, by inflating the cuff to 40 mmHg above the systolic pressure, the plethysmographic waveform disappears indicating that this pressure (systolic pressure+40 mmHg) is sufficient to achieve the full ischemia.

Table 1 summarises the results for all 12 patients. As seen, in 9 patients (75%), the systolic pressure is not enough to maintain ischemia. By increasing the ischemia pressure to 20 mmHg above the systolic pressure there are still 3 patients (25%) whose ischemia is not achieved yet. This indicates that implementing an RIC treatment with a random ischemia pressure and without FCU does not guarantee a complete ischemia even in healthy patients with no prior history or symptoms of heart disease.

TABLE 1
Results of RIC treatment

| | | | Systolic/ | ISCHEMIA PRESSURE | | |
|---|---|---|---|---|---|---|
| | | | Diastolic | | Systolic | Systolic |
| Patient | | | Pressure | Systolic | Pressure + | Pressure + |
| No. | Sex | Age | (mmHg) | Pressure | 20 mmHg | 40 mmHg |
| 1 | M | 39 | 120/78 | Fail | Pass | — |
| 2 | M | 37 | 134/81 | Fail | Fail | Pass |
| 3 | F | 28 | 116/66 | Fail | Pass | — |
| 4 | F | 25 | 103/59 | Pass | — | — |
| 5 | F | 26 | 118/75 | Fail | Fail | Pass |
| 6 | F | 57 | 131/80 | Pass | — | — |
| 7 | F | 24 | 117/66 | Fail | Pass | — |
| 8 | M | 24 | 123/75 | Fail | Pass | — |
| 9 | F | 35 | 135/86 | Pass | — | — |
| 10 | M | 31 | 127/77 | Fail | Pass | — |
| 11 | M | 32 | 122/75 | Fail | Fail | Pass |
| 12 | M | 32 | 128/79 | Fail | Pass | — |

Embodiments of the remote ischemic conditioning system disclosed herein can be used for treatment of one or more of the following conditions: bacterial and fungal diseases; behaviours and mental disorders; blood and lymph conditions; cancers and other neoplasms; digestive system diseases; diseases and abnormalities at or before birth; eye diseases; gland and hormone related diseases; heart and blood diseases; immune system diseases; muscle, bone, and cartilage diseases; nervous system diseases; nutritional and metabolic diseases; respiratory tract (lung and bronchial) diseases; skin and connective tissue diseases; substance related disorders; symptoms and general pathology; urinary tract, sexual organs, and pregnancy conditions; wounds and injuries.

Embodiments of the remote ischemic conditioning system disclosed herein can be applied to remote ischemic conditioning and remote ischemic post-conditioning.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A remote ischemic preconditioning system comprising:
a cuff configured to contract a limb of a subject at a first location;
an actuator connected to the cuff that, when actuated, causes the cuff to contract about the limb of the subject;
a controller that controls the actuator to operate according to a treatment protocol that includes a plurality of ischemia and reperfusion cycles of contracting and releasing the cuff about the limb of the subject;
a first sensor for measuring oxygen saturation level in the blood of the limb;
a second sensor for measuring a pulse property in the limb at a second location;
a feedback control unit having a first feedback layer and a second feedback layer, the feedback control unit being in communication with the controller and configured to receive the oxygen saturation measurement from the first sensor in the first feedback layer and the pulse property from the second sensor in the second feedback layer;
an ischemia pressure unit in communication with the controller and the feedback control unit; configured to receive values of systolic and diastolic pressures;
wherein the feedback control unit, during ischemia cycle, is further configured to:
compare the oxygen saturation level to a first predetermined value and signal the controller to operate the actuator to further inflate the cuff and increase ischemia pressure if the oxygen saturation level is above the predetermined value;
compare the pulse property to a second predetermined value and signal the controller to operate the actuator to further inflate the cuff and increase ischemia pressure if the pulse rate or pulse strength is above the predetermined value;
wherein the feedback control unit, during reperfusion cycle, is further configured to:
compare the oxygen saturation level to a third predetermined value and signal the controller to increase the initial ischemia pressure if the oxygen saturation level is below the predetermined value;
compare the pulse property to a fourth predetermined value and signal the controller to increase the initial ischemia pressure if the pulse rate or pulse strength is below the predetermined value.

2. The system of claim 1, wherein the ischemia pressure unit, during ischemia cycle, is further configured to:
use the value of systolic pressure or an average value of the systolic and diastolic pressures as an initial ischemia pressure and compare the oxygen saturation level to a fifth predetermined value in the first feedback layer and compare the pulse property to a sixth predetermined value in the second feedback layer, and then signal the controller to operate the actuator to deflate the cuff and reduce the pressure in the cuff if the oxygen saturation level in the first feedback layer is below the fifth predetermined value and if the pulse rate or pulse strength in the second feedback layer is below the sixth predetermined value.

3. The system of claim 1, wherein the second location is further along the limb than the first location.

4. The system of claim 3, wherein the limb is an arm or a leg of the subject.

5. The system of claim 3, wherein the limb is an arm of the subject.

6. The system of claim 3, wherein the first location is along the arm of a subject and the second location is on a finger of the subject.

7. The system of claim 1, wherein the pulse property is pulse rate or pulse strength in the limb.

8. The system of claim 1, wherein the first sensor is a pulse oximeter.

9. The system of claim 1, wherein the first sensor is selected from a group consisting of a heart rate sensor, a photoplethysmographic sensor, an ultrasonic flow sensor, an infrared detector, and a near infrared sensor.

10. The system of claim 1, wherein the second sensor is a pulse oximeter.

11. The system of claim 1, wherein the second sensor is selected from a group consisting of a heart rate sensor, a photoplethysmographic sensor, an ultrasonic flow sensor, an infrared detector, and a near infrared sensor.

12. The system of claim 10, wherein the ischemia pressure unit is configured to continually or intermittently communicate with the controller and the feedback control unit.

13. The system of claim 1, wherein the feedback control unit is configured to continually or intermittently communicate with the controller.

14. The system of claim 1, wherein the system further comprises a sphygnamometer.

15. The system of claim 1, wherein the system further comprises a third sensor.

16. The system of claim 15, wherein the third sensor is arranged to measure pulse rate or pulse strength in the ischemic limb and the feedback control unit is configured to receive the pulse rate or pulse strength in the ischemic limb from the third sensor and compare the pulse rate or pulse strength to a seventh predetermined value, wherein, if the pulse rate or pulse strength is above the predetermined value, the feedback control unit signals the controller to operate the actuator to further inflate the cuff.

17. The system of claim 1, further comprising a fourth sensor.

18. The system of claim 17, wherein the fourth sensor is a lactic acid sensor and the feedback control unit is configured to a receive lactic acid level from the fourth sensor and compare the lactic acid level to an eighth predetermined value, wherein, if the lactic acid level is below the predetermined value, the feedback control unit signals the controller to operate the actuator to further inflate the cuff.

19. A method of performing remote ischemic preconditioning of a subject, the method comprising:
providing the system of claim 1;
attaching the cuff about a limb of a subject;
activating the controller to operate according to a treatment protocol that includes a plurality of treatment cycles of contracting and releasing the cuff about the limb of a subject wherein each treatment cycle comprises:
an ischemic duration, during which the controller receives signals from the at least one of the sensors to maintain the cuff contracted about the limb to occlude blood flow through the limb thereby maintaining ischemia, and
a reperfusion duration, during which the cuff is maintained in an at least partially relaxed state to allow blood flow through the limb.
